(19) United States
(12) Patent Application Publication
(10) Pub. No.: US 2012/0183562 A1
(43) Pub. Date: Jul. 19, 2012

(54) SECRETED FXYD PROTEINS EXPRESSED IN RESPONSE TO EPITHELIAL TISSUE DAMAGE, AND USES THEREFOR

(75) Inventors: Gerard Brady, Charlesworth (GB); Shaun Ainsworth, Burnley (GB); Patricia Hurley, London (GB); Christopher Potten, Collwyn Bay (GB); Jeffrey Moore, Manchester (GB)

(73) Assignee: Epistem Limited, Manchester (GB)

(21) Appl. No.: 13/203,193

(22) PCT Filed: Feb. 24, 2010
(86) PCT No.: PCT/US10/25238

§ 371 Date: Apr. 9, 2012

Related U.S. Application Data
(60) Provisional application No. 61/154,978, filed on Feb. 24, 2009.

(51) Int. Cl. A61K 39/395; C07K 17/00; C07K 14/435; G01N 33/53; A61K 39/00; C07H 21/04; A61P 17/00; C07K 16/28; A61K 38/17

(52) U.S. Cl. 424/172.1; 530/389.1; 530/388.2; 530/387.3; 530/391.3; 530/350; 514/17.4; 424/184.1; 536/23.5; 435/7.1

(57) ABSTRACT

Secreted FXYD family proteins are expressed by intestinal mucosa and/or associated tissues to regulate cell production in intestinal crypts in response to tissue damage. Such tissue damage may arise from disease, exposure to injurious chemicals (e.g., due to poisoning, chemotherapy, chemical weapons), or exposure to injurious radiation (e.g., due to nuclear power accidents, radiological weapons, radiation therapy). Because these proteins are secreted in response to epithelial tissue damage, some of them are implicated in tissue repair response or an inflammatory response which prolongs or exacerbates the tissue damage. Examples of these proteins include FXYD 3, FXYD 4, and FXYD 5. Diagnostic methods based upon the role of the FXYD proteins in epithelial tissue damage are disclosed. Also provided are antibodies raised against the FXYD family proteins, a kit for detecting at least an epitope of a polypeptide, a method of producing an antibody raised against an epitope of a polypeptide, and a method of diagnosing damage to an epithelial tissue.

### BACKGROUND OF THE INVENTION

1. Field of the Invention

The invention relates to the identification of secreted proteins in the FXYD family that are expressed by intestinal mucosa and/or associated tissues to regulate cell production in intestinal crypts in response to tissue damage, the use of such FXYD proteins to generate antibodies, and to the use of such FXYD proteins and such antibodies in methods and preparations for the diagnosis of mucosal or other epithelial tissue damage.

2. Description of the Related Art

Throughout most of its length, the mammalian gastrointestinal tract is lined with a mucous membrane formed by an epithelium composed of a single thickness of columnar epithelial cells. In the small intestine this epithelium is molded to cover the villi, the finger-like protrusions of connective tissue which are present in large numbers and which project into the lumen of the gut, while in the surface around and between the villi there are numerous cavities known as crypts (crypts of Lieberkühn) comprising small flask-shaped bags of epithelial cells.

The epithelium covering the villi is non-proliferative, consisting of cells that are fully differentiated and functional, undergoing senescence and loss, primarily by extrusion into the lumen. The cell loss from the villi of the small intestine is extensive, with approximately 10⁹ cells (approximately one gram) being extruded every 5 days in the mouse and probably every 20-30 minutes in the human. This extensive cell loss is balanced in a steady state condition by cell division in the crypts surrounding the base of the villi, with the new cells produced in the crypts migrating out onto the villi to replace the cells being lost from the latter. There are generally about 6 to 10 of these crypts surrounding and serving each villus, and each crypt may serve more than one villus.

As a consequence of the cell replacement process, there is a constant movement of cells from the crypt to the villus. This cell migration can be studied, and the cell velocities measured. At the top of the crypt, the cells generally move with a velocity of between 0.75 and 1.5 cell diameters per hour. Moreover, the movement of cells occurs as vertical columns, with relatively little lateral displacement. In the mouse small intestine, the crypts contain about 250 cells of which 150 are in rapid cell cycle, each dividing approximately twice a day. This makes the small intestine one of the most rapidly proliferating tissues of the body but, in spite of this rapid proliferation, cancers of the small bowel are rare, suggesting that the homeostatic mechanisms are extremely well controlled or that the system is well protected against the propagation and maintenance of genetic errors.

Each crypt contains relatively few stems cells, which give rise to a lineage of transit cells within the crypt which, in turn, give rise to the vertical columns of cells which migrate from the crypts to the tips of the villi, where they are shed. The precise number of stem cells within each crypt is uncertain, but is probably 4-16 in the small intestine of the mouse, with the lower end of the range most likely. A unique feature of this form of tissue organization and growth is the fact that the topographical position of a cell within the tissue can be directly related to its stage of maturation and the position in the crypt from which the cell originated and migrated. By studying the behavior and characteristics of cells at a specific position, features associated with the slowly cycling stem cell population, or with the more rapidly dividing transit cell population, can be investigated. Although the precise position of the stem cells in the tissue is not known for certain, it is known that they are located adjacent to the base of the crypt, either scattered amongst the differentiated Paneth cells which are present over the first 3 cell positions from the base or they are more exclusively distributed at about the 4-5 cell positions from the base of the crypt, immediately above the highest Paneth cell. See, for example, Potten and Loeffler (1987), J. Theoret. Biol. 127:381-391, Potten and Loeffler (1990), Development 110:1001-1020, and Potten (1998), Phil. Trans. Roy. Soc. 353:821-830.

When the tissues of the small intestine are damaged or injured by exposure to cytotoxic agents (e.g., chemotherapeutics) or high doses of radiation (e.g., gamma or X-rays), it is known that the crypts initially shrink in size and overall proliferative activity rapidly declines, leading to a cellular depletion as a consequence of acute cell death (apoptosis) and continued migration of cells onto the villi. However, after a certain period of time, provided the damage is not too severe, regenerative processes restore crypt activity back to the original normal steady state conditions. In fact, during this regenerative phase, crypt size may even enlarge or overshoot beyond the original normal size.

Detailed studies involving examination of the distribution of proliferative changes throughout the different regions of the crypt by cell positional analysis have indicated that the regenerative changes after injury are initiated in the stem cell region towards the base of the crypts and may include not only bursts of renewed proliferation but also reductions in cell cycle time in this region. See, for example, Potten (1991), Univ. Texas MD Anderson Conf. Proceedings, John Wiley, NY, p 155-171; Potten et al. (1988), Int. J. Radiat. Biol., 54(6):1041-1051, and Potten (1990), Int. J. Radiat. Biol., 58(6):925-973.

Medically, damage or injury to the epithelial mucosa by chemotherapy and/or radiotherapy is often a dose-limiting side effect of current cancer therapies. Thus, although chemotherapeutics and radiation exposure can effectively kill rapidly proliferating tumor cells, the rapidly dividing stem cells of the host's mucosal epithelium, including the stem cells of the intestinal crypts, are also damaged or killed, leading to the clinical condition broadly termed “mucositis.” Oral and gastrointestinal mucositis can cause sufficient pain to deter patients from continuing their course of treatment, and/or can cause sufficient wasting due to impaired nutrient absorption as to be life-threatening.

Animal models and assays measuring the survival and proliferation of the epithelial cells of the mammalian intestinal mucosa have been used to test and validate potential treatments to ameliorate chemotherapy and radiotherapy induced mucositis. For example, recombinant human keratinocyte growth factor (KGF, sold as Kepivance® by Amgen, Inc., Thousand Oaks, Calif.), used as a pretreatment, caused an increase in measures of mucosal thickness (villus height and crypt depth), and a 3.5-fold improvement in crypt survival in the small intestine (Farrell et al. (1998), Cancer Res. 58:933-939). In addition, KGF administered on a daily basis to mice exposed to a lethal dose of irradiation increased the thickness of the oral epithelia and afforded protection against the cytotoxic effects of radiation on the stem cells of the crypts (i.e., increased numbers of surviving crypts), which resulted in increased survival (Potten et al. (2001), Cell Growth & Differentiation 12:265-275).

The FXYD family of proteins consists of at least seven mammalian proteins (FXYD 1 through FXYD 8) having a conserved 35-amino acid sequence that begins with the motif PFXYD. Two additional family members have been identified in zebrafish. These small membrane proteins are widely distributed in the body, and highly expressed in tissues that are involved in fluid or solute transport or that are electrically excitable. The FXYD proteins are believed to function as ion transport regulators. Specifically, the FXYD proteins appear to be tissue-specific regulatory subunits of the Na,K-ATPase, and particularly the α1-β isozymes. (See, e.g., Crambert and Geering (2003), “FXYD Proteins: New Tissue-Specific Regulators of the Ubiquitous Na,K-ATPase,” Sci. STKE 2003(166):RE1.)

Prior to the present invention and disclosure, however, the role of FXYD protein expression in the regenerative response of mammalian epithelial tissue to damage or injury, particularly the regenerative response of mammalian mucosal epithelium to damage or injury induced by cytotoxic agents or radiation, was not known in the art.

Therefore, there remains a need in the art for an understanding of the genetic basis for, and the role of FXYD proteins in, the regenerative response to epithelial damage or injury induced by cytotoxic agents or radiation, a need for diagnostic tools for specifically assessing the degree of such damage or injury.

### SUMMARY OF THE INVENTION

The invention depends, in part, upon the identification of secreted proteins in the FXYD family that are expressed by epithelial tissue and/or associated tissues in response to tissue damage. Such tissue damage may arise from infectious disease (e.g., gastric ulcer), non-infectious disease (e.g., inflammatory bowel disease), mechanical or surgical trauma, exposure to injurious chemicals (e.g., due to poisoning, chemotherapy, chemical weapons), or exposure to injurious radiation (e.g., due to nuclear power accidents, radiological weapons, radiation therapy). These FXYD family proteins have been identified by analysis of differential expression in damaged versus undamaged tissues, as well as bioinformatic analysis to identify such secreted proteins.

These secreted FXYD family proteins are, by virtue of their method of identification, necessarily associated with, and therefore diagnostic of, damage to epithelial tissues. Therefore, these secreted FXYD family proteins can be used for the generation of antibodies (or fragments or derivatives thereof) and the antibodies can be used in diagnostic methods to detect or quantify damage to epithelial tissues.

Thus, in one aspect, the invention provides substantially pure preparations of the FXYD family proteins of the invention. These FXYD family protein preparations can be used in immunogenic compositions in methods for producing antibodies that specifically bind the FXYD family proteins of the invention.

In another aspect, the invention provides methods of producing antibodies that specifically bind the FXYD family proteins of the invention. In a related aspect, the invention provides substantially pure preparations of antibodies that specifically bind the FXYD family proteins of the invention. The antibodies can be used in diagnostic methods.

In another aspect the invention provides diagnostic methods, including the detection and quantification of damage to epithelial tissues. In some embodiments, the epithelial damage is caused by chemotherapy or radiotherapy. In some embodiments, the damaged epithelial tissue is mucosal epithelium, as in the oral and intestinal mucosa. These tissues may be present in, for example, the mouth, nose, throat, esophagus, or gastrointestinal tract, in hair follicles, glands (e.g., salivary, liver, breast, prostate, endocrines), and lumenal lining of the lungs. These diagnostic methods include the detection of changes in the levels of secreted FXYD family proteins.

These and other aspects of the invention will be apparent to those of ordinary skill in the art in view of the following detailed description and examples.

### DETAILED DESCRIPTION

The patent, scientific and technical publications referred to herein establish knowledge that was available to those skilled in the art at the time of filing. The entire disclosures of the issued U.S. patents, published and pending patent applications, and scientific and technical publications that are cited herein are hereby incorporated by reference to the same extent as if each was specifically and individually indicated to be incorporated by reference. In the case of any inconsistencies, the present disclosure will prevail.

### Definitions.

All scientific and technical terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art. In addition, in order to more clearly and concisely describe the subject matter which is the invention, the following definitions are provided for certain terms which are used in the specification and appended claims.

As used herein, the term “FXYD family protein” refers to one or more of the human FXYD 3, FXYD 4, or FXYD 5 proteins, or a variant of such a protein (e.g., an allelic variant, homolog or ortholog) which has substantially the same biological activity, or a fragment of such a protein which has substantially the same biological activity, or a mimetic molecule which has substantially the same biological activity, where the biological activity is determined in the context of the assays described in the examples.

As used herein with respect to protein preparations, the term “substantially pure” means a preparation which contains at least 60% (by dry weight) the protein of interest, exclusive of the weight of other intentionally included compounds. In some embodiments, the preparation is at least 75%, at least 90%, or at least 99%, by dry weight the protein of interest, exclusive of the weight of other intentionally included compounds. Purity can be measured by any appropriate method, e.g., column chromatography, gel electrophoresis, or HPLC analysis. If a preparation intentionally includes two or more different proteins of the invention, a “substantially pure” preparation means a preparation in which the total dry weight of the proteins of the invention is at least 60% of the total dry weight, exclusive of the weight of other intentionally included compounds. For such preparations containing two or more proteins of the invention, the total weight of the proteins of the invention can be at least 75%, at least 90%, or at least 99%, of the total dry weight of the preparation, exclusive of the weight of other intentionally included compounds. Thus, if the proteins of the invention are mixed with one or more other proteins (e.g., serum albumin) or compounds (e.g., diluents, detergents, excipients, salts, polysaccharides, sugars, lipids) for purposes of administration, stability, storage, and the like, the weight of such other proteins or compounds is ignored in the calculation of the purity of the preparation.

As used herein, the term “antibody” is intended to embrace naturally produced antibodies, recombinantly produced antibodies, monoclonal antibodies, and polyclonal antibodies, as well as antibody fragments such as Fab fragments, F(ab′)₂ fragments, Fv fragments, and single-chain Fv fragment (scFv). Useful antibodies include all immunoglobulin classes, such as IgM, IgG, IgD, IgE, IgA and their subclasses. Antibodies may be produced by standard methods, well known in the art. See, e.g., Pluckthun (1990), Nature 347:497-498; Huse et al. (1989), Science 246:1275-1289; Chaudhary et al. (1990), Proc. Natl. Acad. Sci. USA 87:1066-1070; Mullinax et al. (1990), Proc. Natl. Acad. Sci. USA 87:8095-8099; Berg et al. (1991), Proc. Natl. Acad. Sci. USA 88:4723-4727; Wood et al. (1990), J. Immunol. 145:3011-3016; and references cited therein.

As used herein, an antibody is said to “specifically bind” a protein of the invention if it is capable of specifically interacting with the protein to non-covalently bind the antibody to the protein. The binding of an antibody to a protein is specific if it distinguishes that protein from any other proteins or other molecules which may be present in a sample. The term “epitope” means a portion of a protein which is recognized and bound by an antibody. An antigen, including the proteins of the invention, may have one or more epitopes. An “immunogenic composition” of an antigen is a composition including an antigen which is capable of inducing an animal to produce at least one antibody that specifically binds to an epitope of that antigen.

As used herein, the terms “increase” and “decrease” mean, respectively, to cause an increase or decrease of at least 5%, as determined by a method and sample size that achieves statistically significance (i.e., p<0.05).

As used herein, the term “statistically significant” means having a probability of less than 5% under the relevant null hypothesis (i.e., p<0.05).

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, . . . , 0.9, 0.99, 0.999, or any other real values≧0 and ≦2, if the variable is inherently continuous.

As used herein, unless specifically indicated otherwise, the word “or” is used in the inclusive sense of “and/or” and not the exclusive sense of “either/or.”

As used herein and in the appended claims, the use of singular forms of words, and the use of the singular articles “a,” “an” and “the,” are intended to include and not exclude the use of a plurality of the referenced term unless the context clearly dictates otherwise.

### General Considerations.

The present invention depends, in part, upon the identification of secreted FXYD family proteins that are expressed by intestinal mucosa and associated tissues (e.g., epithelial, muscle, endothelium, lamina propria, myofibroblasts) and that regulate epithelial stem cells in response to tissue damage. Such tissue damage may arise from infectious disease (e.g., gastric ulcer), non-infectious disease (e.g., inflammatory bowel disease), mechanical or surgical trauma, exposure to injurious chemicals (e.g., due to poisoning, chemotherapy, chemical weapons), or exposure to injurious radiation (e.g., due to nuclear power accidents, radiological weapons, radiation therapy). These FXYD family proteins have been identified by analysis of differential expression in damaged versus undamaged tissues, as well as bioinformatics analysis to identify secreted proteins.

The invention has arisen out of investigations of cell growth and proliferation in certain parts of the mammalian gastrointestinal tract, in particular the mucosal epithelium of the mouse small intestine, following tissue damage or injury such as results from severe doses of radiation (e.g., gamma rays or high energy X-rays or electron beams) or cytotoxic agents (e.g., chemotherapeutics). The mouse gastrointestinal tract includes epithelium that cover villi and numerous cavities known as crypts (crypts of Lieberkühn) comprising small flask-shaped bags of epithelial cells. The crypts in the ileum of mice may contain about 250 cells of which a few are stem cells and about 150 are actively passing through the cell cycle. Cell division in these crypts produce new cells that migrate out onto the villi to replace the cells that cover the villi at a rate of about 2000 cells per day for each villus. The bulk of the proliferating epithelial cells in the ileum have a cell cycle time of between about 11 hours and 19 hours. At locations in the crypt expected to contain the stem cells, the cell cycle time is slower. Exposure to radiation showed that apoptotic cells tended to appear first at these locations where stem cells are expected to be present. Potten (1990), Int. J. Radiat. Biol. 58(6): 925-973.

### Secreted Proteins of the Invention

The FXYD family proteins of the invention include proteins, identified by the methods described above, which are secreted by epithelial and associated tissues in response to epithelial tissue damage arising from radiological and cytotoxic chemical insults (e.g., from radiotherapy or chemotherapy). The proteins were initially identified in a mouse model of epithelial tissue damage that has been validated as a useful model for human tissue response. Booth et al. (2000), Int. J. Cancer 86: 53-5, Farrell et al. (1998), Cancer Res. 58:933-939, and Booth and Potten (2001), J. Nat. Cancer Inst. 29:16-20.

The FXYD family proteins of the invention include, without limitation, the following:

TABLE A

| | Accession | | Accession |
|---|---|---|---|
| Mouse Protein | Number | Human protein | Number(s) |
| FXYD3 | NM_008557 | FXYD3 | NM_001136007 |
| | NP_032583 | | NP_001129479 |
| FXYD4 | NM_033648 | FXYD4 | NM_173160 |
| | NP_387468 | | NP_775183 |
| FXYD5 | BC013340 | FXYD5 | NM_014164 |
| | NP_001104543 | | NP_054883 |

In addition, the FXYD family proteins of the invention include certain fragments of these proteins as well as certain fusion proteins comprising these proteins. For example, various algorithms and computer programs, as described below, can be used to identify antigenic fragments which can be used to produce the antibodies of the invention (see, e.g., http://tools.immuneepitope.org/tools/bcell/iedb_input). Similarly, biologically active fragments can be identified (e.g., based upon structural domains). Fusion proteins can also be produced by standard techniques in order to simplify or facilitate purification by introducing ligand sequences (e.g., poly-His tags, biotin-avidin sequences, maltose-binding protein, c-myc or other epitopes) or to increase antigenicity (e.g., adjuvant sequences such as hemagglutinin, neuraminidase). The FXYD family proteins of the invention can also be labeled by standard techniques, including isotopic (e.g., ¹²⁵I, ³²P, ¹⁵N, ¹³C), fluorescent (e.g., fluorescein, rhodamine) and enzymatic (e.g., alkaline phosphatase, horse radish peroxidase) labels.

### FXYD Family Protein Preparations.

In another aspect, the present invention provides substantially pure preparations of the secreted FXYD family proteins of the present invention. The preparations may be lyophilized preparations, or may be solutions including pharmaceutically acceptable carriers, buffers or diluents. The degree of purification will depend upon the use for which the preparation is intended. For example, the preparation may be a crude extract of cells which naturally or recombinantly express the FXYD family protein for use as an immunogen in animals, or may be a highly purified pharmaceutical formulation suitable for enteral or parenteral administration to a human.

The proteins of the present invention can be isolated from the cells of the intestinal crypts, using standard techniques such as immunoaffinity purification with the antibodies of the invention (see below) or liquid chromatography purification, or can be isolated from the transformed cells of the invention, in which they can be expressed at higher levels and, optionally, as fusion proteins which are more easily isolated and/or purified. An immunoaffinity purification or liquid chromatography purification may entail passing a crude cell extract of colonic or intestinal crypts through liquid chromatography column(s). Isolation from transformed cells may entail expressing cDNA that encodes a protein of the present invention. The proteins can also be isolated by simple diffusion from segments of intestine placed in appropriate saline solutions at about 37° C. These exudates can be subsequently purified by a variety of standardized procedures.

The FXYD family proteins of the invention include the full-length murine and human proteins disclosed in Table A, as well as allelic variants, homologs or orthologs of these proteins.

Fragments of the full-length FXYD family proteins can also be used in certain applications, particularly fragments which include epitopes for the production of antibodies, or structural domains that interact with binding partners for the proteins, such as antibodies or receptors proteins. Potentially useful epitopes of the proteins of the invention can be identified by standard sequence analysis techniques, as described below. Functional equivalents of these proteins can also be produced by standard techniques known to those of skill in the art, including site-directed mutagenesis followed by assays for biological activity, as described below.

In certain embodiments, polypeptides are provided having at least 80%-100% (e.g., 85%, 90% or 95%) amino acid sequence identity with at least a structural domain of a FXYD family protein of the present invention, or with the full-length sequence of a FXYD family protein of the invention. In some embodiments, polypeptides are provided having at least 80%-100% amino acid sequence identity with a FXYD family protein of the invention and which have substantially the same (or greater) biological activity as a protein of the invention as determined by the assays described below. Thus, using routine experimentation and ordinary skill in the art, such mimetic molecules can be produced by standard protein synthesis methods or recombinant protein expression technology, and can be screened for the desired activity using the assays described herein.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient.

Wetting agents, emulsifiers and lubricants, as well as coloring agents, release agents, coating agents, sweetening, flavoring and perfuming agents, preservatives and antioxidants can also be present in the compositions.

### Antibodies.

In another aspect, the present invention provides antibodies that specifically bind the FXYD family proteins of the invention, preparations of such antibodies, and methods of making such antibodies. The antibodies can be polyclonal or monoclonal, and can be made by methods well known in the art. In particular, the invention provides antibodies raised against epitopes having high predicted antigenicity, which therefore will selectively bind to and, thereby, isolate or identify the proteins of the invention.

The antibodies can be raised against the full-length proteins, against fragments of the proteins, or using any epitopes which are characteristic of the proteins and which substantially distinguish them from other proteins. In certain embodiments, the antibodies are raised against epitopes identified by prediction of hydrophobicity, surface probability and antigenic index using standard algorithms or computer programs (e.g., GCG™, Genetics Computer Group, University of Wisconsin Biotechnology Center, Madison, Wisc.; MacVector™, MacVector, Inc., Cary, N.C.). In addition, see http://tools.immuneepitope.org/tools/bcell/iedb_input; and Jameson and Wolf (1988), Comput. Appl. Biosci. 4:181-186.

Useful epitopes of the human FXYD 3, FXYD 4 and FXYD 5 proteins include:

TABLE B

| Human | Accession | | |
|---|---|---|---|
| protein | Number(s) | Residues | Sequences |
| FXYD3 | NP_001129479 | 44-58 | QRDASLPVPGQRSDM |
| | | 78-92 | NDLEDKNSPFYYDWH |
| | | 133-138 | TPPLITP |
| FXYD4 | NP_775183 | 17-32 | LEANDPFANKDDPFYY |
| | | 62-79 | KCKSSQKQHSPVPEKAIP |
| FXYDS | NP_054883 | 21-35 | GQTLKDTTSSSSADS |
| | | 53-62 | ELQPTSPTPT |
| | | 64-76 | PADETPQPQTQTQ |

Immunogen preparations of the proteins of the invention can be produced from crude extracts, from proteins or peptides substantially purified from cells which naturally or recombinantly express them or, for small immunogens, by chemical peptide synthesis. The immunogen preparations can also be in the form of a fusion protein in which one portion is chosen for its adjuvant properties and/or its ability to facilitate purification (e.g., polyhistidine) and the other portion is derived from a protein of the invention.

The immunogen preparations may comprise a FXYD family protein of the invention, or a fragment thereof, in a pharmaceutically acceptable carrier or diluent, with or with out an adjuvant.

As a general matter, monoclonal antibodies that specifically bind the FXYD family proteins of the invention can be produced by first injecting a mouse, rabbit, goat or other suitable animal with an immunogen in a pharmaceutically acceptable carrier or diluent. Carrier proteins or adjuvants can be utilized, and booster injections (e.g., bi- or tri-weekly over 8-10 weeks) can be employed as necessary. After allowing for development of a humoral response, the animals are sacrificed and their spleens are removed and resuspended in an appropriate buffer (e.g., phosphate buffered saline). The spleen cells serve as a source of lymphocytes, some of which will produce antibodies of the appropriate specificity. These cells are then fused with an immortalized cell line (e.g., a myeloma), and the products of the fusion are plated into tissue culture wells in the presence of a selective agent (e.g., HAT). The wells are serially screened and replated, selecting cells making a useful antibody each time. Typically, several screening and replating procedures are carried out until the wells contain single clones which are positive for antibody production. Monoclonal antibodies produced by such clones can be purified by standard methods such as affinity chromatography using Protein A Sepharose, by ion-exchange chromatography, or by variations and combinations of these techniques.

The antibodies of the invention can be polyclonal or monoclonal, or can be antibody fragments, including Fab fragments, F(ab′)₂ fragments, Fv fragments, and single chain Fv fragments (scFv). In addition, after identifying useful antibodies by the methods of the invention, recombinant antibodies can be generated, including any of the antibody fragments listed above, and including chimeric and/or humanized antibodies based upon non-human antibodies to the FXYD family proteins of the invention. In light of the present disclosure of the FXYD family proteins of the invention, one of ordinary skill in the art can produce the above-described antibodies by any of a variety of standard means. For an overview of antibody techniques, see, e.g., Antibody Engineering, 2nd Ed., Borrebaeck, ed., Oxford University Press, Oxford (1995); Rubinstein et al. (2003), Anal Biochem 314:294-300; Traggia et al. (2004), Nat. Med. 10(8):871-875; and Lanzavecchia et al. (2006), Immunol. Rev., 2 11:303-309. Antibody phage display strategies also can be employed to identify useful antibodies, including chimeric or humanized antibodies, according to protocols known in the art (e.g., Krebs et al. (2001), J. Immunol. Methods 254: 67-84; Rauchenberger et al. (2003), J. Biol. Chem. 278:381 94-38205; Magnussen et al. (2005), Cancer Res. 65:2712-2721; U.S. Pat. No. 6,753,136).

The antibodies of the invention can be used in a variety of applications. For example, antibodies can be used in assays to detect the presence or level of the FXYD family proteins of the invention in body fluids (e.g., blood, lymph, the mucosal layer of the intestine), or in assays to measure the presence or level of expression of the proteins of the invention in particular cells (e.g., epithelial cells, including intestinal mucosal cells, and further including stem cells of the intestinal mucosa). Techniques employing the antibodies of the invention include Western blotting to identify cells expressing the FXYD family proteins of the invention, immunocytochemistry or immunofluorescence techniques to establish the cellular or extracellular location of the proteins of the invention, and ELISA techniques to detect the presence or quantify the FXYD family proteins of the invention in a sample.

The antibodies of the invention can be bound to or conjugated with other compounds or materials for diagnostic uses. For example, they can be coupled to labels such as radionuclides, fluorescent compounds (e.g., rhodamine), or enzymes for imaging. The labels can be bound to the antibodies covalently or non-covalently.

In another aspect, the invention provides kits for detecting at least an epitope of a protein of the invention. The kits include an antibody that specifically binds a protein of the invention and a means for detecting the antibody. The means for detecting the antibody can be a detectable label bound to the antibody or secondary antibodies for detecting the primary antibodies (e.g., a labeled goat anti-rabbit-Ig antibody as a secondary antibody for detecting a rabbit antibody that specifically binds a protein of the invention).

### Nucleic Acid Preparations.

In one aspect, the present invention provides preparations of isolated nucleic acid molecules encoding the secreted FXYD family proteins of the invention or useful fragments thereof (e.g., antigenic fragments, functional domains). These nucleic acids can be used to synthesize the FXYD family proteins of the invention or as probes or primers for the diagnostic methods of the invention.

The nucleic acid molecules can be DNA or RNA molecules, hybrid DNA-RNA molecules, or nucleic acid analogs. The nucleic acid analogs can include modified bases (e.g., 2′-halo-2′-deoxynucleosides) and/or modified internucleoside linkages (e.g., peptide nucleic acids, phosphorothioate linkages). The nucleic acids can be sense molecules corresponding to all or a portion of the gene sequence encoding a protein of the invention, or can be antisense molecules which are complementary to all or a portion of a gene sequence encoding a protein of the invention. The nucleic acids can be derived from, or correspond to, genomic DNA or cDNA, or can be synthetic molecules based upon a protein sequence and the genetic code (e.g., synthetic nucleic acids which reflect the codon usage preferences in the host cells used in an expression system).

In some embodiments, the nucleic acids comprise the entire coding sequence of a gene encoding a FXYD family protein of the invention. Such nucleic acids can be used to produce genetic constructs for transformation of cells, or for in vitro transcription and translation systems. Such nucleic acids can also be used as probes in hybridization assays or immobilized in hybridization arrays to detect sequences in samples, such as mRNA or cDNA molecules that encode a protein of the invention.

In other embodiments, subsets of the nucleic acid sequences are provided for use as primers for nucleic acid amplification reactions, as probes in hybridization assays or arrays to detect sequences in samples, or as probes to distinguish normal or wild-type sequences from abnormal or mutant sequences. In these embodiments, the nucleic acids of the invention comprise 10-60 nucleotides, typically at least 10, 12, 14, 16 or 18 consecutive nucleotides selected from a nucleic acid sequence that encodes a FXYD family protein of the invention. Depending upon the nature of the application, it can be preferable to choose sequences which will have unique targets, or which are expected to have unique targets, within a sample being probed or amplified. Thus, for example, sequences which are longer and sequences which do not include frequently repeated elements (for example, polyadenylation signals) are more likely to be uniquely represented within any given sample. For purposes of choosing primers for amplification reactions, sequences of at least 15 nucleotides, and typically 18-25 nucleotides, are used. For purposes of using a probe, the probe can have a detectable label bound to the probe or a secondary nucleic acid probe for detecting the first probe (e.g., labeled secondary nucleic acid which specifically hybridizes to the isolated nucleic acid). These nucleic acids may include a nucleotide sequence that hybridizes to at least a portion of a sequence coding a FXYD family protein of the invention under stringent hybridization conditions. Such conditions include hybridizations employing a wash step of 1.0×SSC at 65° C., and equivalents thereof. More stringent conditions can include wash steps of 0.5×SSC, 0.2×SSC, or even 0.1×SSC. Other conditions equivalent in stringency are well known in the art. See, e.g., Ausubel et al., eds. (1989), Current Protocols in Molecular Biology, Vol. I, John Wiley & Sons, Inc., New York.

In other embodiments, the nucleic acids of the invention encode polypeptides including an amino acid sequence of at least 10 amino acid residues in length, up to and including the length of the complete protein as initially synthesized or as post-translationally processed into intermediate or mature forms.

### Genetic Constructs.

In another aspect, the present invention provides genetic constructs comprising nucleic acids of the invention. In one series of embodiments, coding sequences (e.g., the entire coding region, sequences encoding structural domains, sequences encoding epitopes, or sequences encoding useful primers or probes) are operably joined to an endogenous or exogenous regulatory region to form an expression construct. Useful regulatory regions for these purposes include the endogenous regulatory region, constitutive promoter sequences (e.g., CMV, SV40, EF2), and inducible promoter sequences (e.g., lacZ, tet). Many useful vector systems are commercially available. For example, useful bacterial vectors include, but are not limited to, pQE70, pQE60, pQE-9 (Qiagen, Valencia, Calif.), pBluescript II™ (Stratagene, La Jolla, Calif.), and pTRC99a, pKK223-3, pDR540 and pRIT2T (Pharmacia, Piscataway, N.J.), pTrc (Amann et al. (1988), Gene 69:301-315) and pET 11d (Studier et al. (1990), Methods Enzymol. 185:60-89). Examples of vectors for expression in yeast include pYepSec1 (Baldari et al. (1987), EMBO J. 6:229-234), pMFa (Kurjan et al. (1982), Cell 30:933-943), pJRY88 (Schultz et al. (1987), Gene 54:113-123), and pYES2 (Invitrogen Corporation, San Diego, Calif.). The proteins can also be expressed in insect cells (e.g., Sf 9 cells) using, for example, baculovirus expression vectors including, but not limited to, pAc vectors (Smith et al. (1983), Mol. Cell Biol. 3:2156-2165) and pVL vectors (Lucklow et al. (1989), Virology 170:31-39). Examples of mammalian expression vectors include, but are not limited to, pCDM8 (Seed (1987), Nature 329:840) and pMT2PC (Kaufman et al. (1987), EMBO J. 6:187-195). Other useful eukaryotic vectors include, but are not limited to, pXT1, pSG5 (Stratagene, La Jolla, Calif.), and pSVK3, pBPV, pMSG, and PSVLSV40 (Pharmacia, Piscataway, N.J.).

In another series of embodiments, the nucleic acid coding sequences can be joined to regulatory regions and exogenous coding sequences to form a genetic construct or fusion vector which encodes a fusion protein. In some embodiments, the coding sequences can be joined to exogenous coding sequences that confer new and useful properties to the fusion protein. For example, fusion vectors and fusion proteins can be useful to increase the expression of the protein, to increase the solubility of the protein, or to aid in the purification of the protein (e.g., by providing a ligand sequence for affinity purification). A proteolytic cleavage site can be introduced at the junction of the FXYD family protein of the invention and the fusion moiety so that the FXYD family protein of the invention can easily be separated from the fusion moiety. Typical fusion expression vectors include pGEX (Smith et al. (1988), Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

### Transformed Cell Lines.

In another aspect, the present invention provides cell lines transformed with the nucleic acid molecules of the invention. Such cell lines can simply propagate these nucleic acids (e.g., when transformed with cloning vectors) or can express the polypeptides encoded by these nucleic acids (e.g., when transformed with expression vectors). Such transformed cell lines can be used to produce the FXYD family proteins of the invention and fragments of the invention.

The transformed cells can be produced by introducing into a cell an exogenous nucleic acid or nucleic acid analog which replicates within that cell, that encodes a polypeptide sequence which is expressed in that cell, and/or that is integrated into the genome of that cell so as to affect the expression of a genetic locus. The transformation can be achieved by any of the standard methods referred to in the art as transformation, transfection, transduction, electroporation, ballistic injection, and the like. The method of transformation is chosen to be suitable to the type of cells being transformed and the nature of the genetic construct being introduced into the cells.

Useful cell lines for transformation include bacterial cells (e.g., Escherichia coli), yeast cells (e.g., Saccharomyces cerevisiae), insect cells (e.g., Drosophila melanogaster Schneider cells), nematode cells (e.g., Caenorhabditis elegans), amphibian cells (e.g., Xenopus oocytes), rodent cells (e.g., Mus musculus (e.g., murine 3T3 fibroblasts), Rattus rattus, Chinese Hamster Ovary cells (e.g., CHO-K1)), and human cells (e.g., human skin fibroblasts, human embryonic kidney cells (e.g., HEK-293 cells), COS cells).

The cells can be transformed according to any method known in the art appropriate to the cell type being transformed. Appropriate methods include those described generally in, e.g., Sambrook et al. (1989), Molecular Cloning: A Laboratory Manual, Cold Spring Harbor Laboratories, New York; and Davis et al. (1986), Basic Methods in Molecular Biology, Elsevier. Particular methods include calcium phosphate co-precipitation (Graham et al. (1973), Virol. 52:456-467), direct micro-injection into cultured cells (Capecchi (1980), Cell 22:479-488), electroporation (Shigekawa et al. (1988), BioTechniques 6:742-751), liposome-mediated gene transfer (Mannino et al. (1988), BioTechniques 6:682-690), lipid-mediated transduction (Felgner et al. (1987), Proc. Natl. Acad. Sci. USA 84:7413-7417), and nucleic acid delivery using high-velocity microprojectiles (Klein et al. (1987), Nature 327:70-73).

### Diagnostic Methods.

In another aspect, the invention provides diagnostic methods in which the presence or level of synthesis or secretion of a FXYD family protein of the invention is assayed to detect the presence or level of epithelial tissue damage or injury, such as mucosal epithelial damage or injury induced by cytotoxic agents (e.g., chemotherapeutics) or radiation exposure (e.g., radiotherapy), or to detect the presence or level of epithelial cell proliferative response to such injury. A level of protein synthesis or secretion significantly higher or lower than a standard reference level (e.g., the level in normal, healthy individuals who have not been subjected to epithelial tissue damage or injury) can be used as an indication that the subject has sustained epithelial tissue damage or injury, or that an epithelial cell proliferative response to such injury has been induced. Alternatively, or in addition, a series of standard reference levels can be determined (e.g., levels typically resulting from certain dosages of a chemotherapeutic, or exposure to certain levels of radiation), and the level detected in a subject can be used as an indication that the subject has sustained a degree of epithelial tissue damage or injury associated with the reference level.

In some embodiments, the antibodies of the invention, which specifically bind the FXYD family proteins of the invention, can be used to test samples from blood, lymph, fluids from the oral cavity (sputum), tear ducts, peritoneal and pleural cavities, lung exudates, urine, or stools, or from biopsies of the oral or gastrointestinal mucosa, the bulb of cells at the end of plucked hairs, or nipple aspirates of a subject to determine the presence or level of synthesis or secretion of the FXYD family proteins of the invention. Techniques for measuring the presence or levels of the FXYD family proteins of the invention include Western blotting, immunocytochemistry or immunofluorescence techniques, and ELISA techniques.

In other embodiments, when biopsies of the tissue are available, mRNA expression can be tested to ascertain the presence or level of expression of the FXYD family proteins of the invention, as described in the assays below. As with detecting protein secretion, levels of expression can be compared to one or more standard reference levels and used as an indication that tissue damage has occurred, or that an epithelial cell proliferative response has been induced. The amount of mRNA expression can be measured by hybridization to a whole genome microarray, or by a selective array including a probe for the gene of interest and, optionally, additional probes for calibration. Such a measurement may entail producing cDNA from the total mRNA produced in a cell of the biopsied tissue (e.g., a crypt cell), synchronously amplifying the cDNA, using the cDNA to hybridize to the microarray, and measuring the amount of hybridization. Alternatively, the amount of mRNA expression can be measured by RT-qPCR, as described below.

These and other methods of measuring the amount of FXYD family protein secretion or mRNA expression will be apparent to one of ordinary skill in the art based upon the nucleic acid and protein sequences disclosed herein.

### EXAMPLE 1

### Determination of mRNA Expression Levels of FXYD Family Member Genes in the Lower and Upper Segments of Colonic Crypts in Mice using Microarrays.

mRNA Expression Profiling of Individual Intestinal Crypts. Mice were sacrificed by cervical dislocation and intestines excised from groups of 3 normal male BDF₁ mice (first generation hybrid of C56/BL6 female×DBA2 males, Harlan UK Ltd, Oxon, UK) aged 10-12 weeks. Crypts were isolated from sections of colon using methods described by Booth et al (1995) In Vitro 31: 234-43 Epith.Cell Biol 4:76-86, Evous et al (1992) J Cell Sci 101: 219-31. Individual colonic crypts were cut in half using a sapphire knife with the aid of an inverted microscope. Total RNA was prepared from each upper and lower crypt segment (RNAqueous®-96, Applied Biosystems/Ambion, Austin, Tex.). cDNA was generated from mRNA and amplified synchronously using methods described by Brady et al. (1990), Methods Mol. Cell. Biol. 2:17-25; Al-Taher et al. (2000), Yeast 17(3):201-10; and Brady (2000), Yeast 17(3):211-7. The relative abundance of nucleic acid sequences was determined by hybridisation of cDNA to whole genome mouse microarrays (Agilent, Wokingham, UK).

FXYD Gene Family Expression in Normal Colonic Crypts. The results of mRNA profiling of lower and upper crypts from 3 individual mice are reported in Table 1. The top portion of Table 1 displays the raw data from the mice, whereas the bottom portion shows the ratio of expression between the upper and lower crypt cells. FXYD4 expression levels were approximately 2.6-fold higher in the upper segment of colonic crypt; FXYD3 and FXYD5 expression levels were more similar in both segments of crypts. Expression of other FXYD family member genes (FXYD1, FXYD2, FXYD6, and FXYD7) was not detected in crypts, which is consistent with the literature (Geering (2006), Am. J. Physiol. Renal Physiol. 290(2):F241-50).

TABLE 1
Untreated Mice
Raw Data (Arbitrary Fluorescence Units)

| | Mouse no. | | | | | |
|---|---|---|---|---|---|---|
| | 1 | | 2 | | 3 | |
| | Lower | Upper | Lower | Upper | Lower | Upper |
| FXYD3 | 34 | 33 | 113 | 40 | 42 | 20 |
| FXYD4 | 1159 | 2143 | 395 | 1293 | 206 | 1825 |
| FXYD5 | 626 | 592 | 305 | 390 | 384 | 498 |

Ratio of Gene Expression in Upper/Lower Crypts

| | Mouse no. | | | | Mouse no. | | | |
|---|---|---|---|---|---|---|---|---|
| | 1 | 2 | 3 | Ave* | 1 | 2 | 3 | Ave* |
| | Ratio of Lower/Upper | | | | Ratio of Upper/Lower | | | |
| FXYD3 | 1.1 | 2.8 | 2.1 | 2.5 | 0.9 | 0.4 | 0.5 | 0.4 |
| FXYD4 | 0.5 | 0.3 | 0.1 | 0.4 | 1.8 | 3.3 | 8.8 | 2.6 |
| FXYD5 | 1.1 | 0.8 | 0.8 | 0.8 | 0.9 | 1.3 | 1.3 | 1.3 |

*Ave = Mean of two of three closest values determined by ratios.

FXYD Expression Levels in the Regenerative Phase of Damage Response. To determine the potential role of FXYD family genes in regenerating intestinal tissues in response to damage induced by chemotherapy, mice were subjected to chemotherapy and mRNA levels profiled from individual colonic crypts.

Chemotherapy Damage Response Model (Regenerative Phase). Groups of 3 male BDF₁ mice aged 10-12 weeks were given 2 intraperitoneal injections 6 hours apart of 5-fluorouracil (5-FU, Sigma-Aldrich Corp., Poole, UK) at 400 mg per kg body weight. Mice were sacrificed 24 hours after the second 5-FU treatment.

The results of mRNA profiling of lower and upper crypts from mice subjected to the chemotherapy damage response model are reported in Table 2. The top portion of Table 2 displays the raw data from the mice, whereas the bottom portion shows the ratio of expression between the lower and upper crypt cells (left portion) and, inversely, the ratio of expression between the upper and lower crypt cells (right portion). FXYD4 expression levels were 54.7-fold higher in the upper segment of colonic crypts, compared to approximately 2.6-fold higher in untreated mice; FXYD3 expression was approximately 3.5-fold higher in the lower segment of crypts for treated mice, compared to an average of 2.5-fold higher in untreated mice. No differences were observed for FXYD5 expression in untreated or treated mice.

TABLE 2
Chemotherapy-Treated Mice
Raw Data (Arbitrary Fluorescence Units)

| | Mouse no. | | | | | |
|---|---|---|---|---|---|---|
| | 1 | | 2 | | 3 | |
| | Lower | Upper | Lower | Upper | Lower | Upper |
| FXYD3 | 179 | 63 | 100 | 59 | 109 | 26 |
| FXYD4 | 56 | 3213 | 87 | 4456 | 865 | 3471 |
| FXYD5 | 437 | 412 | 519 | 305 | 1024 | 272 |

Ratios of Gene Expression in Upper and Lower Crypts

| | Mouse no. | | | | Mouse no. | | | |
|---|---|---|---|---|---|---|---|---|
| | 1 | 2 | 3 | Ave* | 1 | 2 | 3 | Ave* |
| | Ratio of Lower/Upper | | | | Ratio of Upper/Lower | | | |
| FXYD3 | 2.8 | 1.7 | 4.1 | 3.5 | 0.4 | 0.6 | 0.2 | 0.3 |
| FXYD4 | 0.0 | 0.0 | 0.2 | 0.0 | 57.9 | 51.5 | 4.0 | 54.7 |
| FXYD5 | 1.1 | 1.7 | 3.8 | 1.4 | 0.9 | 0.6 | 0.3 | 0.4 |

*Ave = Mean of two of three closest values

Differential mRNA expression of FXYD genes in upper and lower colonic crypts during the regenerative phase of damage response indicates this family of genes may coordinate cell production.

### EXAMPLE 2

### Confirmation by RT-qPCR of mRNA Expression Changes of FXYD Family Genes in the Lower and Upper Segments of Colonic Crypts in Mice

To confirm the microarray results in Example 1 and more accurately quantify expression levels, real-time quantitative PCR (RT-qPCR) was performed on the representative global cDNA samples. Oligonucleotide probes (Table 3) were fluorescently labeled (SYBR® Green qPCR Core kit, Eurogentec, Liege, Belgium) and RT-qPCR was performed (ABI PRISM® 7000 Sequence Detection System, Applied Biosystems, Inc., Carlsbad, Calif.).

TABLE 3
Oligonucleotide Probes for Mouse FXYD Genes
FXYD3
Associated sequence accession number NM_008557
5′-TTCTCCTCCCCTCCTGACACT-3′ (sense)
5′-CTGAACAAAGAGCCTGCTACCA-3′ (anti-sense)
FXYD4
Associated sequence accession number NM_033648
5′ -GGCAGGTTTTTGACAACTTTCTG-3′ (sense)
5′ -CAGTATGTTCTAGGGAAGCCATCCT-3′ (anti-sense)
FXYD5
Associated sequence accession number BC013340
5′ -TGATCAACATGAAAGAATCCTGAAA-3′ (sense)
5′ -GGTGGGAAGACCATTAGCCTTA-3′ (anti-sense)

The results of profiling mRNA expression in lower and upper crypts from 3 individual normal mice are reported in Table 4. FXYD3 expression levels were approximately 2.2-fold higher in the lower segment of colonic crypts; FXYD4 expression levels were approximately 2.6-folder higher in the upper segment of the colonic crypts. No differential expression pattern was observed with FXYD5.

TABLE 4
Untreated Mice
Raw data (PCR Units, Linear)

| | Mouse no. | | | | | |
|---|---|---|---|---|---|---|
| | 1 | | 2 | | 3 | |
| | Lower | Upper | Lower | Upper | Lower | Upper |
| FXYD3 | 107 | 48 | 187 | 85 | 91 | 105 |
| FXYD4 | 53 | 125 | 12 | 34 | 5 | 60 |
| FXYD5 | 91 | 97 | 34 | 19 | 15 | 43 |

Ratios of Gene Expression in Upper and Lower Crypts

| | Mouse no. | | | | Mouse no. | | | |
|---|---|---|---|---|---|---|---|---|
| | 1 | 2 | 3 | Ave* | 1 | 2 | 3 | Ave* |
| | Ratio of Lower/Upper | | | | Ratio of Upper/Lower | | | |
| FXYD3 | 2.2 | 2.2 | 0.9 | 2.2 | 0.4 | 0.5 | 1.1 | 0.4 |
| FXYD4 | 0.4 | 0.4 | 0.1 | 0.4 | 2.3 | 2.8 | 11.5 | 2.6 |
| FXYD5 | 0.9 | 1.9 | 0.4 | 0.6 | 1.1 | 0.5 | 2.8 | 0.8 |

*Ave = Mean of two of three closest values

### FXYD Expression Levels in Regenerative Phase of Damage Response.

To confirm the microarray results in Example 1 and more accurately quantify expression levels, real-time quantitative PCR (RT-qPCR) was performed on the representative global cDNA samples prepared from colonic crypts in the regenerative phase of the response to damage induced by chemotherapy.

The results of mRNA profiling of lower and upper crypts from 3 individual mice are reported in Table 5. FXYD4 expression levels were approximately 73.1-fold higher in the upper segment of colonic crypts, compared to approximately 2.6-fold higher in untreated mice; FXYD3 expression levels were approximately 3.1-fold higher in the lower segment of colonic crypts, compared to approximately 2.2-fold higher in untreated mice; and FXYD5 expression levels were approximately 11.2-fold higher in lower crypts, compared to similar levels in untreated mice.

RT-qPCR results confirmed the microarray results in Example 1 and showed that expression levels of FXYD3, FXYD4, and FXYD5 genes increased during the regenerative phase of the response to damage induced by chemotherapy. These results indicate that FXYD genes are involved in regulating cell production in colonic crypts.

TABLE 5
Chemotherapy-Treated Mice
Raw Data (PCR Units, Linear)

| | Mouse no. | | | | | |
|---|---|---|---|---|---|---|
| | 1 | | 2 | | 3 | |
| | Lower | Upper | Lower | Upper | Lower | Upper |
| FXYD3 | 158 | 51 | 229 | 46 | 108 | 34 |
| FXYD4 | 0 | 141 | 2 | 184 | 15 | 574 |
| FXYD5 | 28 | 43 | 109 | 8 | 504 | 64 |

Ratios of Gene Expression in Upper and Lower Crypts

| | Mouse no. | | | | Mouse no. | | | |
|---|---|---|---|---|---|---|---|---|
| | 1 | 2 | 3 | Ave* | 1 | 2 | 3 | Ave* |
| | Ratio of Lower/Upper | | | | Ratio of Upper/Lower | | | |
| FXYD3 | 3.1 | 4.9 | 3.1 | 3.1 | 0.3 | 0.2 | 0.3 | 0.3 |
| FXYD4 | 0.0 | 0.0 | 0.0 | 0.0 | 4693.7 | 108.1 | 38.1 | 73.1 |
| FXYD5 | 0.6 | 14.5 | 7.8 | 11.2 | 1.6 | 0.1 | 0.1 | 0.1 |

*Ave = Mean of two of three closest values

### Equivalents.

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize, or be able to ascertain using no more than routine experimentation, many equivalents to the specific embodiments of the invention described specifically herein. Such equivalents are intended to be encompassed in the scope of the appended claims.

SEQUENCE LISTING
SEQ ID NO: 1 (mouse FXYD3, Accession Number NP_032583.1)

| | |
|---|---|
| 1 | mqevvlsllv llaglptlda ndpenkndpf yydwyslrvg glicagilca |
| | lgiivlmsgk |
| 61 | ckckfrqkps hrpgegppli tpgsahnc |

SEQ ID NO: 2 (human FXYD3, Accession Number NP_001129479.1)

| | |
|---|---|
| 1 | mgrgysgalq arggleeple rglrgpsftq splhgaaaay lsaqrdaslp |
| | vpgqrsdmqk |
| 61 | vtlgllvfla gfpvldandl edknspfyyd whslqvggli cagvlcamgi |
| | iivmsakckc |
| 121 | kfgqksghhp getpplitpg saqs |

SEQ ID NO: 3 (mouse FXYD4, Accession Number NP_387468.1)

| | |
|---|---|
| 1 | meeitcafll llaglpalea sdpvdkdspf yydweslqlg glifggllci |
| | agiamalsgk |
| 61 | ckcrrthkps slpgkatpli ipgsantc |

SEQ ID NO: 4 (human FXYD4, Accession Number NP_775183.1)

| | |
|---|---|
| 1 | mervtlalll lagltalean dpfankddpf yydwknlqls glicggllai |
| | agiaavlsgk |
| 61 | ckckssqkqh spvpekaipl itpgsattc |

SEQ ID NO: 5 (mouse FXYDS, Accession Number NP_001104543.1)

| | |
|---|---|
| 1 | mslssr1c11 tivalilpsr gqtpkkptsi ftadqtsatt rdnvpdpdqt |
| | spgvqttpli |
| 61 | wtreeatgsq taaqtetqql tkmatsnpvs dpgphtsskk gtpaysriep |
| | lspsknfmpp |
| 121 | sylehpldsn ennpfyyddt tlrkrgllva avlfitgill ltsgkcrqls qfclnrhr |

SEQ ID NO: 6 (human FXYDS, Accession Number NP_054883.3)

| | |
|---|---|
| 1 | mspsgrlcll tivglilptr gqtlkdttss ssadstimdi qvptrapdav |
| | ytelqptspt |
| 61 | ptwpadetpq pqtqtqqleg tdgplvtdpe thkstkaahp tddtttlser |
| | pspstdvqtd |
| 121 | pqtlkpsgfh eddpffydeh tlrkrgllva avlfitgill ltsgkcrqls rlcrnrcr |

What is claimed is:

1. A method for diagnosing damage to an epithelial tissue in a subject, comprising:
measuring in a sample from said subject an indicator of the presence or level of expression of at least one polypeptide selected from the group consisting of FXYD 3, FXYD 4 and FXYD 5;
comparing said measured indicator to a reference level; and
diagnosing the tissue damage if said indicator is increased/decreased relative to the reference level.

2. A method for detecting a proliferative response in an epithelial tissue in a subject, comprising:
measuring in a sample from said subject an indicator of the presence or level of expression of at least one polypeptide selected from the group consisting of FXYD 3, FXYD 4 and FXYD 5;
comparing said measured indicator to a reference level; and
identifying a proliferative response in said tissue if said indicator is increased/decreased relative to the reference level.

3. The method of claim 1 or 2 wherein said indicator is an indicator of the level of mRNA encoding the polypeptide.

4. The method of claim 1 or 2 wherein said indicator is an indicator of the level of the polypeptide secreted from an epithelial tissue.

5. The method of claim 4, wherein measuring said indicator comprises:
contacting the sample with an antibody that specifically binds the polypeptide, thereby forming a complex of the antibody and the polypeptide, and
measuring the amount of complexes formed.

6. A method as in claim 1, wherein the damage to the epithelial tissue is induced by chemotherapy or radiotherapy.

7. A method as in claim 2, wherein the proliferative response is induced by chemotherapy or radiotherapy.

8. A substantially pure antibody preparation comprising an antibody that specifically binds an epitope of a polypeptide selected from the group consisting of FXYD 3, FXYD 4 and FXYD 5.

9. The substantially pure antibody preparation of claim 8 wherein said epitope has high predicted antigenicity.

10. The substantially pure antibody preparation of claim 8 wherein said antibody is a monoclonal antibody.

11. The substantially pure antibody preparation of claim 8 wherein said antibody is an antibody fragment selected from the group consisting of an Fab fragment, an F(ab′)₂ fragment, an Fv fragment, and a single-chain Fv fragment (scFv).

12. A kit for detecting damage to an epithelial tissue comprising:
an antibody raised against an epitope of a polypeptide selected from the group consisting of FXYD 3, FXYD 4 and FXYD 5; and
a means for detecting said antibody.

13. The kit of claim 12 wherein said antibody comprises a detectable label bound thereto.

14. The kit of claim 12 wherein said kit further comprises a labeled secondary antibody which specifically binds to said antibody.

15. A substantially pure protein preparation comprising at least one polypeptide selected from the group consisting of FXYD 3, FXYD 4, FXYD 5, or a fragment or mimetic thereof.

16. A substantially pure protein preparation comprising at least one polypeptide comprising at least one epitope of high predicted antigenicity of a protein selected from the group consisting of FXYD 3, FXYD 4 and FXYD 5.

17. The substantially pure protein preparation of claim 15 or 16, wherein the preparation is lyophilized.

18. The substantially pure protein preparation of claim 15 or 16, wherein the preparation further comprises a pharmaceutically acceptable carrier.

19. The substantially pure protein preparation of claim 15 or 16, wherein the preparation further comprises an adjuvant.

20. The substantially pure protein preparation of claim 15 or 16, wherein the preparation is formulated for parenteral administration.

21. The substantially pure protein preparation of claim 15 or 16, wherein the preparation is formulated for enteral administration.

22. The substantially pure protein preparation of claim 15 or 16, wherein the preparation is formulated for topical administration.

23. A method for producing an antibody that specifically binds an epitope of a polypeptide selected from the group consisting of FXYD 3, FXYD 4 and FXYD 5, said method comprising:
administering to an animal at least one polypeptide of claim 15 or 16;
allowing for development of a humoral response to said polypeptide in said animal; and
recovering said antibody from said animal.

24. An isolated nucleic acid comprising a nucleotide sequence that encodes at least one polypeptide selected from the group consisting of FXYD 3, FXYD 4, FXYD 5, or a fragment or mimetic thereof.

25. An isolated nucleic acid comprising a nucleotide sequence that encodes at least one polypeptide comprising at least one epitope of high predicted antigenicity of a protein selected from the group consisting of FXYD 3, FXYD 4 and FXYD 5.
